(19) United States
(12) Patent Application Publication
(10) Pub. No.: US 2005/0119304 A1
(43) Pub. Date: Jun. 2, 2005

(54) Urea derivatives

(76) Inventors: Takeshi Yura, Aichi-ken (JP); Muneto Mogi, Nara-ken (JP); Yuka Ikegami, Kyoto-shi (JP); Tsutomu Masuda, Aichi-ken (JP); Toshio Kokubo, Nara-ken (JP); Klaus Urbahns, Lund (SE); Nagahiro Yoshida, Kyoto-fu (JP); Makiko Marumo, Nara-ken (JP); Masahiro Shiroo, Cambridge (GB); Masaomi Tajimi, Aichi-ken (JP); Keisuke Takeshita, Kagawa-ken (JP); Toshiya Moriwaki, Nara-ken (JP); Yasuhiro Tsukimi, Hyogo-ken (JP)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/499,788

(22) PCT Filed: Dec. 13, 2002
(86) PCT No.: PCT/EP02/14215

(30) Foreign Application Priority Data
Dec. 26, 2001 (JP) 2001-395031

(51) Int. Cl. A61K 31/4709; A61K 31/428; A61K 31/405

(52) U.S. Cl. 514/314; 514/367; 514/419

(57) ABSTRACT

A medicament which contains a urea derivative or a salt thereof as an active ingredient is disclosed. The medicament has an excellent activity as VR1 antagonist and useful for the prophylaxis and treatment of diseases associated with VR1 activity, in particular for the treatment of urge urinary incontinence, overactive bladder, chronic pain, neuropathic pain, postoperative pain, rheumatoid arthritic pain, neuralgia, neuropathies, algesia, nerve injury, ischaemia, neurodegeneration, stroke, incontinence and/or inflammatory disorders.

### DETAILED DESCRIPTION OF INVENTION

1. Technical Field

The present invention relates to a urea derivative which is useful as an active ingredient of pharmaceutical preparations. The urea derivative of the present invention has a vanilloid receptor (VR1) antagonistic activity, and can be used for the prophylaxis and treatment of diseases associated with VR1 activity, in particular for the treatment of urge urinary incontinence, overactive bladder, chronic pain, neuropathic pain, postoperative pain, rheumatoid arthritic pain, neuralgia, neuropathies, algesia, nerve injury, ischaemia, neurodegeneration, stroke, incontinence and/or inflammatory disorders.

2. Background

Vanilloid compounds are characterized by:the presence of vanillyl group or a functionally equivalent group. Examples of several vanilloid compounds or vanilloid receptor, modulators are vanillin (4-hydroxy-3-methoxy-benzaldehyde), guaiacol (2-methoxy-phenol), zingeroone (4-/4-hydroxy-3-methoxyphenyl/-2-butanon), eugenol-(2-methoxy4-/2-propenyl/phenol), and capsaicin (8-methy-N-vanillyl-6-nonene-amide).

Among others, capsaicin, the main pungent ingredient in “hot” chili peppers, is a specific neurotoxin that desensitizes C-fiber afferent neurons. Capsaicin interacts with vanilloid receptors (VR1), which are predominantly expressed in cell bodies of dorsal root ganglia (DRG) or nerve endings of afferent sensory fibers including C-fiber nerve endings [Tominaga M, Caterina M J, Malmberg A B, Rosen T A, Gilbert H Skinner K, Raumann B E, Basbaum A I, Julius D: The cloned capsaicin receptor integrates multiple pain-producing stimuli. Neuron. 21: 531-543, 1998]. The VR1 receptor was recently cloned [Caterina M J, Schumacher M A, Tominaga M, Rosen T A, Levine J D, Julius D: Nature 389: 816-824, (1997)] and identified as a nonselective cation channel with six transmembrane domains that is structurally related to the TRP (transient receptor potential) channel family. Binding of capsaicin to VR1 allows sodium, calcium and possibly potassium ions to flow down their concentration gradients, causing initial depolarization and release of neurotransmitters from the nerve terminals. VR1 can therefore be viewed as a molecular integrator of chemical and physical stimuli that elicit neuronal signals in a pathological conditions or diseases.

There are abundant of direct or indirect evidence that shows the relation between VR1 activity and diseases such as pain, ischaemia, and inflammatory (e.g., WO 99/00115 and 00/50387). Further, it has been demonstrated that VR1 transduce reflex signals that are involved in the overactive bladder of patients who have damaged or abnormal spinal reflex pathways [De Groat W C: A neurologic basis for the overactive bladder. Urology 50 (6A Suppl): 36-52, 1997]. Desensitisation of the afferent nerves by depleting neurotransmitters using VR1 agonists such as capsaicin has been shown to give promising results in the treatment of bladder dysfunction associated with spinal cord injury and multiple sclerosis. [(Maggi C A: Therapeutic potential of capsaicin-like molecules—Studies in animals and humans. Life Sciences. 51: 1777-1781, 1992) and (DeRidder D; Chandiramani V; Dasgupta P; VanPoppel H; Baert L; Fowler C J: Intravesical capsaicin as a treatment for refractory detrusor hyperreflexia: A dual center study with long-term followup. J. Urol. 158: 2087-2092, 1997)].

It is anticipated that antagonism of the VR1 receptor would lead to the blockage of neurotransmitter release, resulting in prophylaxis and treatment of the condition and diseases associated with VR1 activity.

It is therefore expected that antagonists of the VR1 receptor can be used for prophylaxis and treatment of the condition and diseases including chronic pain, neuropathic pain, postoperative pain, rheumatoid arthritic pain, neuralgia, neuropathies, algesia, nerve injury, ischaemia, neurodegeneration, stroke, incontinence, inflammatory disorders, urge urinary incontinence (UUI), and/or overactive bladder.

WO 2000/50387 discloses the compounds having a vanilloid agonist activity represented by the general formula: 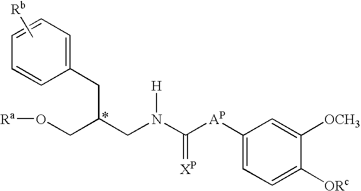
wherein;
X^{P} is an oxygen or sulfur atom;
A^{P} is —NHCH₂— or —CH₂—;
R^{a} is a substituted or unsubstituted C₁₋₄ alkyl group, or R^{a1}CO—;
   wherein
   R^{a1} is an alkyl group having 1 to 18 carbon atoms, an alkenyl group having 2 to 18 carbon atoms, or substituted or unsubstituted aryl group having 6 to 10 carbon atoms;
R^{b} is a hydrogen atom, an alkyl group having 1 to 6 carbon atoms, an alkoky group having 1 to 6 carbon atoms, a haloalkyl group having 1 to 6 carbon atoms or a halogen atom;
R^{C} is a hydrogen atom, an alkyl group having 1 to 4 carbon atom, an aminoalkyl, a diacid monoester or α-alkyl acid; and
the asteric mark * indicates a chiral carbon atom, and their pharmaceutically acceptable salts.

WO 2000/61581 discloses amine derivatives represented by the general formula: 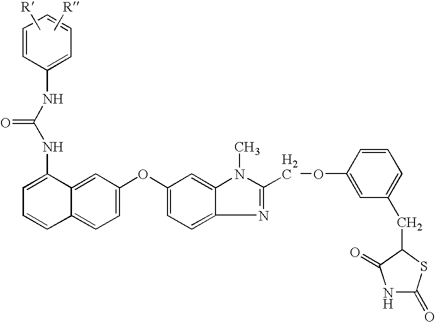
wherein
(R′, R″) represent (F, F), (CF₃, H), or (iPr, iPr)
as useful agents for diabetes, hyperlipemia, arteriosclerosis and cancer.

WO 2000/75106 discloses the compounds represented by the general formula: 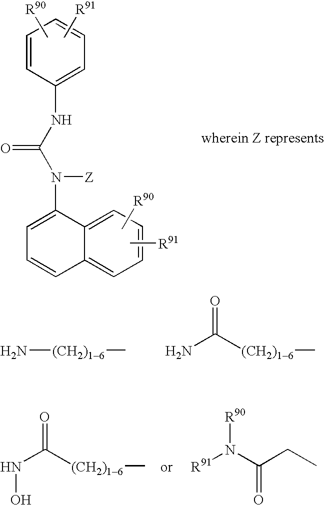
in which
R⁹⁰ is hydrogen, C₁₋₁₂ alkyl, C₃₋₈ cycloalkyl, or the like, and
R⁹¹ is amino-C₁₋₆ alkyl, aminocarbonyl-C₁₋₆ alkyl, or hydroxy-aminocarbonyl C₁₋₆ alkyl; and
R⁹⁰ and R⁹¹ are independently selected from the group consisting of H, C₁₋₆ alkyl, C₁₋₆ alkylthio, C₁₋₆ alkoxy, fluoro, chloro, bromo, iodo, and nitro;
as useful agents for treating MMP-mediated diseases in mammals.

However, none of these reference discloses simple derivatives having pharmaceutical activity.

The development of a compound having effective VR1 antagonistic activity and the use of such compound for the prophylaxis and treatment of diseases associated with VR1 activity, in particular, for the treatment of urge urinary incontinence and/or overactive bladder have been desired.

### SUMMARY OF THE INVENTION

This invention is to provide a medicament comprising an urea derivative of the formula (I), its tautomeric or stereoisomeric form, or a salt thereof as an active ingredient: 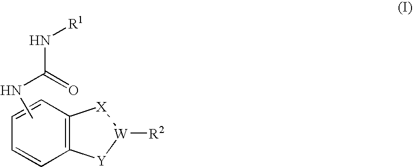
wherein
R¹ is C₁₋₆ alkyl substituted by phenyl or thienyl (in which said phenyl or thienyl are substituted by R¹¹, R¹², and R¹³), C₃₋₈ cycloalkyl optionally fused by benzene, thienyl, quinolyl, carbazolyl of which N—H is substituted by N—R¹¹, 1,2-oxazolyl substituted, by R¹¹, naphthyl substituted by R¹⁴ and R¹⁵, phenyl substituted by R¹¹, R¹² and R¹³, phenyl fused by C₄₋₈ cycloalkyl or saturated or unsaturated C₄₋₈ hetero cyclic ring having one or two hetero atoms selected from the group consisting of N, O, S, and SO₂,
wherein said cycloalkyl and heterocyclic ring are optionally substituted by R¹¹,
in which
R¹¹, R¹² and R¹³ are different or identical and represent hydrogen, halogen, oxo, nitro, carboxyl, C₁₋₆ alkyl optionally substituted by hydroxy or mono-, di-, or tri-halogen, carbamoyl, C₁₋₆ alkylcarbamoyl, C₁₋₆ alkoxy optionally substituted: by mono-, di-, or tri-halogen, C₁₋₆ alkoxycarbonyl, amino, C₁₋₆ alkyl-amino, di(C₁₋₆ alkyl)amino, morpholino, benzyl, phenoxy, mono-, di-, or tri-halogen substituted phenoxy, C₁₋₆ alkylthio, C₁₋₆ alkanoyl, C₁₋₆ alkanoylamino, C₁₋₆ alkyl substituted 4,5-dihydro-1,3-oxazolyl, 1,2,3-thiadiazolyl, phenyl optionally substituted by one to three substituents,
   in which the substituents are each different or identical and selected from the group consisting of hydrogen, halogen, C₁₋₆ alkoxy, C₁₋₆ alkyl, C₁₋₆ alkanoyl, and carboxy, or
   the substituent represented by the formula —SO₂—N—R¹¹¹
   wherein
   R¹¹¹ represents hydrogen, 5-methyl-isoxazole, or 2,4 di-methylpyrimidine;
R¹⁴ is hydrogen, hydroxy, or C₁₋₆ alkoxy;
R¹⁵ is hydrogen, hydroxy, or C₁₋₆ alkoxy;
X, Y, and W are different or identical represent C, CH, CH₂, C(O), N, NH, S, O, SO or SO₂;
the dashed line between X and W represents a single bond or a double bond;
R² is selected from the group consisting of hydrogen, methyl, hydroxy, mercapto, trifluoromethyl, and methylthio, or
   is absent;
with the proviso that if the bond between X—W is a double,
X is N or CH;
W is N or C; and
Y is selected from the group consisting of NH, S, O, CH₂, SO, and SO₂;
with the proviso that when W is N, R² is absent;
if the bond between X—W is a single,
X and Y independently represent CH₂, CO, NH, S, O, SO, or SO₂; and
W is N, CH, S, O, SO or SO₂;
with the proviso that when W is S, O, SO or SO₂, R² is absent.

The urea derivative of formula (I), its tautomeric and stereoisomeric form, and salts thereof surprisingly shows excellent VR1 antagonistic activity. They are, therefore, suitable especially for the prophylaxis and treatment of diseases associated with VR1 activity, in particular for the treatment of urge urinary incontinence and/or overactive bladder.

This invention is also to provide a method for treating or preventing a disorder or disease associated with VR1 activity in a human or animal subject, comprising administering to said subject a therapeutically effective amount of the urea derivative shown in the formula (I), its tautomeric or stereoisomeric form, or a physiologically acceptable salt thereof.

Further this invention is to provide a use of the urea derivative shown in the formula (I), its tautomeric or stereoisomeric form, or a physiologically acceptable salt thereof in the preparation of a medicament. Preferably, said medicament is suitable for treating or preventing a disorder or disease associated with VR1 activity.

In another preferable embodiment, the urea derivative of formula (I) are those wherein;
R¹ is 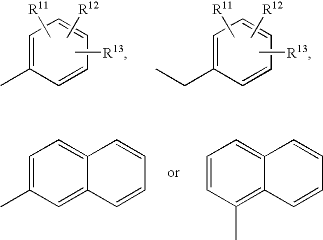
   wherein
R¹¹, R¹², and R¹³ are different or identical and: represent hydrogen, halogen, nitro, carboxyl, C₁₋₆ alkyl optionally substituted by hydroxy or mono-, di-, or tri-halogen, C₁₋₆ alkoxy optionally substituted by mono-, di-, or tri-halogen, C₁₋₆ alkoxycarbonyl, carbamoyl, C₁₋₆ alkylcarbamoyl, amino, C₁₋₆ alkylamino, di(C₁₋₆ alkyl)amino, morpholino, phenyl, benzyl, phenoxy, mono-, di-, or tri-halogen substituted phenoxy, mono-, di-, or tri-halogen substituted phenyl, C₁₋₆ alkylthio, C₁₋₆ alkanoyl, C₁₋₆ alkanoylamino, or the substituent represented by the formula —S₂—N—R¹¹¹
wherein
R¹¹¹ is hydrogen, 5-methyl-isoxazole, or 2,4-dimethyl-pyrimidine.

In another preferable embodiment, the urea derivative of formula (I) are those wherein;
R¹ is 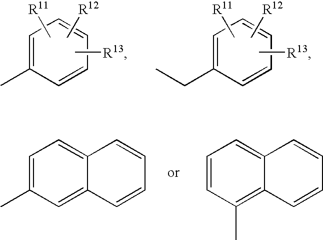
R¹¹, R¹², and R¹³ are different or identical and represent hydrogen, fluoro, chloro, bromo, methyl, isopropyl, methoxy, nitro, ethoxy-carbonyl, phenyl, phenoxy, 4-chlorophenyl, methylthio, acetyl, or trifluoromethyl.

In another preferable embodiment, the urea derivative of formula (I) are those wherein; 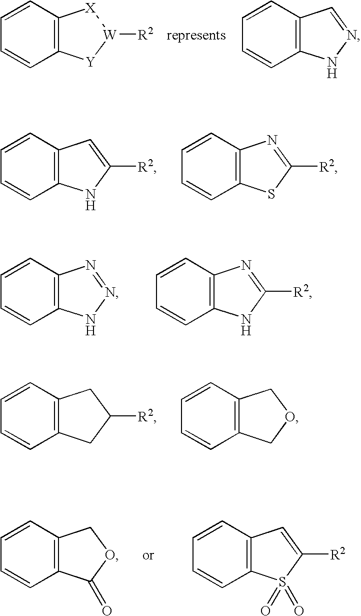
wherein
R² is hydrogen, methyl, hydroxy, mercapto, trifluoromethyl, or methyl-thio.

Most preferably, said urea derivative of the formula (I) is selected from the group consisting of:
N-[4-chloro-3-(trifluoromethyl)phenyl]-N′-(1H-indazol-5-yl)urea;
N-[4-chloro-3-(trifluoromethyl)phenyl]-N′-(1H-indol-7-yl)urea;
N-[4-chloro-3-(trifluoromethyl)phenyl]-N′-(1H-indol-4-yl)urea;
N-[4-chloro-3-(trifluoromethyl)phenyl]-N′-[2-(trifluoromethyl)-1H-benzimidazol-4-yl]urea;
N-(4-bromobenzyl)-N′-(1H-indol-7-yl)urea;
N-[4-chloro-3-(trifluoromethyl)phenyl]-N′-(1,1-dioxido-1-benzothien-6-yl)urea;
N-(1,3-benzothiazol-6-yl)-N′-[4-chloro-3-(trifluoromethyl)phenyl]urea;
N-[4-chloro-3-(trifluoromethyl)phenyl]-N′-(2-methyl-1,3-benzothiazol-5-yl)urea;
N-(2-methyl-1,3-benzothiazol-5-yl)-N′-(3-methylphenyl)urea;
N-(4-fluorophenyl)-N′-(2-methyl-1,3-benzothiazol-5-yl)urea;
N-(2-methyl-1,3-benzothiazol-5-yl)-N′-[3-(trifluoromethyl)phenyl]urea;
N-(2-methyl-1,3-benzothiazol-5-yl)-N′-(4-phenoxyphenyl)urea;
N-(4-bromophenyl)-N′-(2-methyl-1,3-benzothiazol-5-yl)urea;
N-(2-methyl-1,3-benzothiazol-5-yl)-N′-(2-naphthyl)urea;
N-(3,4-dichlorophenyl)-N′-(2-methyl-1,3-benzothiazol-5-yl)urea;
N-(2,4-difluorophenyl)-N′-(2-methyl-1,3-benzothiazol-5-yl)urea;
N-(3-chloro-4-methylphenyl)-N′-(2-methyl-1,3-benzothiazol-5-yl)urea;
N-[2-chloro-5-(trifluoromethyl)phenyl]-N′-(2-methyl-1,3-benzothiazol-5-yl)urea;
N-(4-isopropylphenyl)-N′-(2-methyl-1,3-benzothiazol-5-yl)urea;
N-(2-methyl-1,3-benzothiazol-5-yl)-N′-(1-naphthyl)urea;
N-(1H-indol-4-yl)-N′-[3-(trifluoromethyl)phenyl]urea;
N-(1,1′-biphenyl-3-yl)-N′-(1H-indol-4-yl)urea;
N-[4-chloro-3-(trifluoromethyl)phenyl]-N′-(2-methyl-1H-benzimidazol-4-yl)urea;
N-(2-methyl-1H-benzimidazol-4-yl)-N′-(4-phenoxyphenyl)urea;
N-(1H-indol-4-yl)-N′-(1-naphthyl)urea;
N-(3,4-dichlorophenyl)-N′-(1H-indol-4-yl)urea;
N-(3-chloro-4-methylphenyl)-N-(1H-indol-4-yl)urea;
N-(1H-indol-4-yl)-N′-(4-isopropylphenyl)urea;
N-(4-fluorophenyl)-N′-(1H-indazol-5-yl)urea;
N-[2-chloro-5-(trifluoromcthyl)phenyl]-N′-(1H-indol-4-yl)urea;
ethyl 3-{[(1H-indol-4-ylamino)carbonyl]amino}benzoate; and
N-(4-bromobenzyl)-N′-(1H-indol-4-yl)urea.

Preferably, the medicament of the present invention further comprise one or more pharmaceutically acceptable excipients.

The medicament having at least one urea derivative of the formula (I), its tautomeric and stereoisomeric form, and salts thereof is effective for treating or preventing a disease selected from the group consisting of urge urinary incontinence, overactive bladder, chronic pain, neuropathic pain, postoperative pain, rheumatoid arthritic pain, neuralgia, neuropathies, algesia, nerve injury, ischaemia, neurodegeneration and/or stroke, since the diseases also relate to VR1 activity.

Alkyl per se and “alk” and “alkyl” in alkoxy, alkanoyl, alkylthio, alkylamino, alkyl-aminocarbonyl alkylaminosulphonyl, alkylsulphonylamino, alkoxycarbonyl, alkoxy-carbonylamino, alkylcarbamoyl and alkanoylamino represent a linear or branched alkyl radical having generally 1 to 6, preferably 1 to 4 and particularly preferably 1 to 3 carbon atoms, representing illustratively and preferably methyl, ethyl, n-propyl, isopropyl, tert-butyl, n-pentyl and n-hexyl.

Alkoxy illustratively and preferably represents methoxy, ethoxy, n-propoxy, isopropoxy, tert-butoxy, n-pentoxy and n-hexoxy.

Alkanoyl illustratively and preferably represents acetyl and propanoyl.

Alkylamino represents an alkylamino radical having one or two (independently selected) alkyl substituents, illustratively, and preferably representing methylamino, ethylamino, n-propylamino, isopropylamino, tert-butylamino, n-pentylamino, n-hexyl-amino, N,N-dimethylamino, N,N-diethylamino, N-ethyl-N-methylamino, N-methyl-N-n-propylamino, N-isopropyl)-N-n-propylamino, N-t-butyl-N-methylamino, N-ethyl-N-n-pentylamino and N-n-hexyl-N-methylamino.

Alkylaminocarbonyl or alkylcarbamoyl represents an alkylaminocarbonyl radical having one or two (independently selected) alkyl substituents, illustratively and preferably representing methylaminocarbonyl, ethylaminocarbonyl, n-propylamino-carbonyl, isopropylamino-carbonyl, tert-butoxylaminocarbonyl, n-pentylamino-carbonyl, n-hexylaminocarbonyl, N,N-dimethylaminocarbonyl, N,N-diethylamino-carbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentylamino-carbonyl and N-n-hexyl-N-methylaminocarbonyl.

Alkoxycarbonyl illustratively and preferably represents methoxycarbonyl, ethoxy-carbonyl, n-propoxycarbonyl, isopropoxycarbonyl, tert-butoxycarbonyl, n-pentoxy-carbonyl and n-hexoxycarbonyl. Alkoxycarbonylamino illustratively and preferably represents methoxycarbonylamino, ethoxycarbonylamino, n-propoxycarbonylamino, isopropoxycarbonylamino, tert-butoxycarbonylamino, n-pentoxycarbonylamino and n-hexoxycarbonylamino.

Alkanoylamino illustratively and preferably represents acetylamino and ethyl-carbonylamino.

Halogen represents fluorine, chlorine, bromine and iodine.

Aryl per se and in arylamino and in arylcarbonyl represents a mono- to tricyclic aromatic carbocyclic radical having generally 6 to 14 carbon atoms, and more preferably from 6-10 carbon atoms, optionally substituted with one or more substituents. Examples of aryl radicals include, but are not limited to phenyl, naphthyl, indenyl, azulenyl, fluorenyl, anthracenyl, biphenyl, fluorenonyl and the like.

Heterocyclic ring refers to a 3- to 15-membered ring radical which consists of carbon atoms and from one to five heteroatoms selected from the group consisting of nitrogen, oxygen and sulfur. The heterocyclic ring radical may be a monocyclic, bicyclic or tricyclic ring system, which may include fused or bridged ring and may be partially or fully saturated or aromatic. Examples of such rings include, but are not limited to thienyl, benzothienyl, furanyl, benzofuranyl, pyrazinyl, pyrazolyl, pyridazinyl, pyridyl, pyrimidinyl, pyrrolyl, isothiazolyl, thiazolyl, oxazolyl, isoxazolyl, triazolyl, tetrazolyl, imidazolyl, thiadiazolyl, benzothiadiazolyl, oxoadiazolyl, benzothiazolyl, indolyl, carbazolyl, quinolinyl, isquinolinyl, benzo-dioxolyl, indazolyl, indazolinolyl and the like

### EMBODIMENT OF THE INVENTION

The compound of the formula (I) of the present invention can be, but not limited to be, prepared by either of the methods [A], [B] and [C] below. In some embodiments, one or more of the substituents, such as amino group, carboxyl group, and hydroxyl group of the compounds used as starting materials or intermediates are advantageously protected by a protecting group known to those skilled in the art. Examples of the protecting groups are described in “Protective Groups in. Organic Synthesis (3rd Edition)” by Greene and Wuts, John Wiley and Sons, New York 1999.

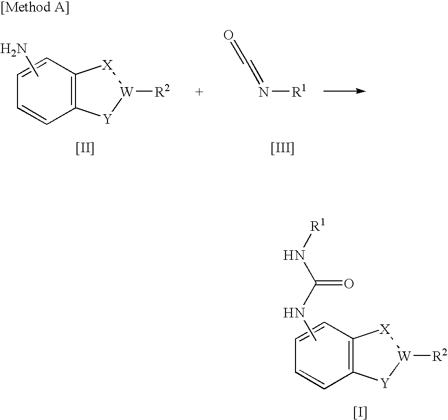

The compound [I] wherein R¹, R², X, Y, and W are the same as defined above, can be prepared by the reaction of an amine derivative formula [II] (wherein R², X, Y, and W are the same as defined above)and isocyanate of the formula [III] (wherein R¹ is the same as defined above).

The reaction may be carried out in a solvent including, for instance, ethers, such as dioxane, and tetrahydrofuran; aromatic hydrocarbons such as benzene, toluene and xylene; nitrites such as acetonitrile; amides such as dimethylformamide, (DMF) and dimethylacetamide; sulfoxides such as dimethyl sulfoxide, and others.

The reaction temperature can be optionally set depending on the compounds to be reacted. The reaction temperature is usually, but not limited to, about 30° C. to 100° C. The reaction may be conducted for, usually, 30 minutes to 48 hours and preferably 1 to 24 hours.

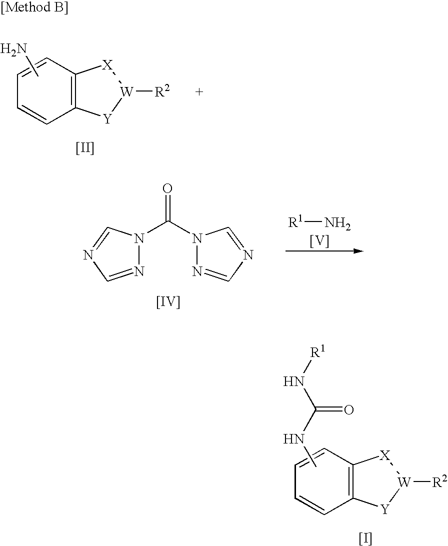

Alternatively, the compound [I] wherein R¹, R², X, Y and W are the same as defined above, can also be prepared by (1) reacting a amine derivative formula [II] (wherein R², X, Y, and W are the same as defined above)and 1,1′-carbonyldi(1,2,4-triazole) (CDT)[IV], and (2) adding amine represented by the formula R¹—NH₂ [V](wherein R¹ is the same as defined above) to the reaction mixture. The reaction (1) may be carried out in a solvent including, for instance, ethers, such as dioxane, and tetra-hydrofuran; aromatic hydrocarbons such as benzene, toluene and xylene; nitriles such as acetonitrile; amides such as dimethylformamide (DMF) and dimethylacetamide; sulfoxides such as dimethyl sulfoxide, and others.

The reaction temperature can be optionally set depending on the compounds to be reacted. The reaction temperature is usually, but not limited to, about 20° C. to 50° C.

The reaction may be conducted for, usually, 30 minutes to 10 hours and preferably 1 to 24 hours.

The reaction (2) may be carried out in a solvent including, for instance, ethers, such as dioxane, and tetrahydrofuran; aromatic hydrocarbons such as benzene, toluene and xylene; nitriles such as acetonitrile; amides such as dimethylformamide (DMF) and dimethylacetamide; sulfoxides such as dimethyl sulfoxide, and others.

The reaction temperature can be optionally set depending on the compounds to be reacted. The reaction temperature is usually, but not limited to, about 30° C. to 100° C. The reaction may be conducted for, usually, 1 hour to 48 hours and preferably 2 to 24 hours.

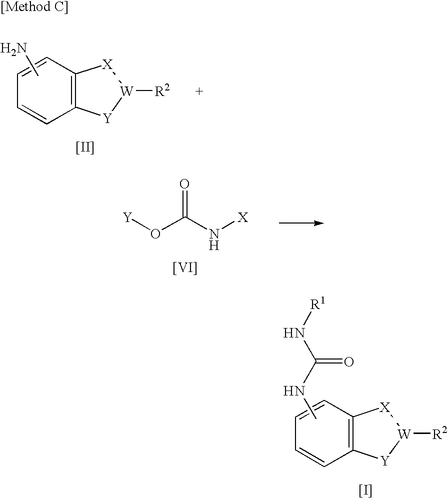

Alternatively, the compound [I] (wherein R¹, R², X, Y, and W are the same as defined above) can be prepared by reacting an amine derivative formula [II] (wherein R², X, Y and W are the same as defined above)and carbamate of the formula [VI] (wherein X is the same as defined above and Y represents phenyl).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitriles such as acetonitrile; amides such as N,N-dimethylformamide (DMF), N,N-dimethylacetamide (DMAC) and N-methylpyrrolidone(NMP); urea such as 1,3-dimethyl-2-imidazolidinone (DMI); sulfoxides such as dimethylsulfoxide (DMSO); and others.

The reaction temperature can be optionally set depending on the compounds to be reacted. The reaction temperature is usually, but not limited to, about 20° C. to 100° C. The reaction may be conducted for, usually, 30 minutes to 40 hours and preferably 1 to 24 hours.

The amine derivatives formula [II], Isocyanates [III], CDT [IV], amines [V], and carbamates [VI] are commercially available or can be prepared by the use of known techniques or by method described in the examples.

When the compound shown by the formula (I) or a salt thereof has tautomeric isomers and/or stereoisomers (e.g., geometrical isomers and conformational isomers), each of their separated isomer and mixtures are also included in the scope of the present invention.

When the compound shown by the formula (I) or a salt thereof has an asymmetric carbon in the structure, their optically active compounds and racemic mixtures are also included in the scope of the present invention.

Typical salts of the compound shown by the formula (I) include salts prepared by reaction of the compounds of the present invention with a mineral or organic acid, or an organic or inorganic base. Such salts are known as acid addition and base addition salts, respectively.

Acids to form acid addition salts include inorganic acids such as, without limitation, sulfuric acid, phosphoric acid, hydrochloric acid, hydrobromic acid, hydriodic acid and the like, and organic acids, such as, without limitation, p-toluenesulfonic acid, methanesulfonic acid, oxalic acid, p-bromophenylsulfonic acid, carbonic acid, succinic acid, citric acid, benzoic acid, acetic acid, and the like.

Base addition salts include those derived from inorganic bases, such as, without limitation, ammonium hydroxide, alkaline metal hydroxide, alkaline earth metal hydroxides, carbonates, bicarbonates, and the like, and organic bases, such as, without limitation, ethanolamine, triethylamine, tris(hydroxymethyl)aminomethane, and the like. Examples of inorganic bases include sodium hydroxide, potassium hydroxide, potassium carbonate, sodium carbonate, sodium bicarbonate, potassium bicarbonate, calcium hydroxide; calcium carbonate, and the like.

The compound of the present invention or a salts thereof, depending on its substituents, may be modified to form lower alkylesters or known other esters; and/or hydrates or other solvates. Those esters, hydrates, and solvates are included in the scope of the present invention.

The compound of the present invention may be administered in oral forms, such as, without limitation normal and enteric coated tablets, capsules, pills, powders, granules, elixirs, tinctures, solution, suspensions, syrups, solid and liquid aerosols and emulsions. They may also be administered in parenteral forms, such as without limitation, intravenous, intraperitoneal, subcutaneous, intramuscular, and the like forms, well-known to those of ordinary skill in the pharmaceutical arts. The compounds of the present invention can be administered in intranasal form via topical use of suitable intranasal vehicles, or via transdermal routes, using transdermal delivery systems well-known to those of ordinary skilled in the art.

The dosage regimen with the use of the compounds of the present invention is selected by one of ordinary skill in the arts, in view of a variety of factors, including, without limitation, age, weight, sex, and medical condition of the recipient, the severity of the condition to be treated, the route of administration, the level of metabolic and excretory function of the recipient, the dosage form employed, the particular compound and salt thereof employed.

The compounds of the present invention are preferably formulated prior to administration together with one or more pharmaceutically-acceptable excipients. Excipients are inert substances such as, without limitation, carriers, diluents, flavoring agents, sweeteners, lubricants, solubilizers, suspending agents, binders, tablet disintegrating agents and encapsulating material.

Yet another embodiment of the present invention is pharmaceutical formulation comprising a compound of the invention and one or more pharmaceutically-acceptable excipients that are compatible with the other ingredients of the formulation and not deleterious to the recipient thereof. Pharmaceutical formulations of the invention are prepared by combining a therapeutically effective amount of the compounds of the invention together with one or more pharmaceutically-acceptable excipients therefore. In making the compositions of the present invention, the active ingredient may be mixed with a diluent, or enclosed within a carrier, which may be in the form of a capsule, sachet, paper, or other container. The carrier may serve as a diluent, which may be solid, semi-solid, or liquid material which acts as a vehicle, or can be in the forms of tablets, pills, powders, lozenges, elixirs, suspensions, emulsions, solutions, syrups, aerosols, ointments, containing, for example, up to 10%, by weight of the active compound, soft and hard gelatin capsules, suppositories, sterile injectable solutions and sterile packaged powders.

For oral administration, the active ingredient may be combined with an oral, and non-toxic, pharmaceutically-acceptable carrier, such as, without limitation, lactose, starch, sucrose, glucose, sodium carbonate, mannitol, sorbitol, calcium carbonate, calcium phosphate, calcium sulfate, methyl cellulose, and, the like; together with, optionally, disintegrating agents, such as, without limitation, maize, starch, methyl cellulose, agar, bentonite, xanthan gum, alginic acid, and the like; and optionally, binding agents, for example, without limitation, gelatin, natural sugars, beta-lactose, corn sweeteners, natural and synthetic gums, acacia, tragacanth, sodium alginate, carboxymethylcellulose, polyethylene glycol, waxes, and the like; and, optionally, lubricating agents, for example, without limitation, magnesium stearate, sodium stearate, stearic acid, sodium oleate, sodium benzoate, sodium acetate, sodium chloride, talc, and the like.

In powder forms, the carrier may be a finely divided solid which is in admixture with the finely divided active ingredient. The active ingredient may be mixed with a carrier having binding properties in suitable proportions and compacted in the shape and size desired to produce tablets. The powders and tablets preferably contain from about 1 to about 99 weight percent of the active ingredient which is the novel composition of the present invention. Suitable solid carriers are magnesium carboxymethyl cellulose, low melting waxes, and cocoa butter.

Sterile liquid formulations include suspensions, emulsions, syrups and elixirs. The active ingredient can be dissolved or suspended in a pharmaceutically acceptable carrier, such as sterile water, sterile organic solvent, or a mixture of both sterile water and sterile organic solvent.

The active ingredient can also be dissolved in a suitable organic solvent, for example, aqueous propylene glycol. Other compositions can be made by dispersing the finely divided active ingredient in aqueous starch or sodium carboxymethyl cellulose solution or in a suitable oil.

The formulation may be in unit dosage form, which is a physically discrete unit containing a unit dose, suitable for administration in human or other mammals. A unit dosage form can be a capsule or tablets, or a number of capsules or tablets. A “unit dose” is a predetermined quantity of the active compound of the present invention, calculated to produce the desired therapeutic effect, in association with one or more excipients. The quantity of active ingredient in a unit dose may be varied or adjusted from about 0.1 to about 1000 milligrams or more according to the particular treatment involved.

Typical oral dosages of the present invention, when used for the indicated effects, will range from about 0.01 mg /kg/day to about 100 mg/kg/day, preferably from 0.1 mg/kg/day to 30 mg/kg/day, and most preferably from about 0.5 mg/kg/day to about 10 mg/kg/day. In the case of parenteral administration, it has generally proven advantageous to administer quantities of about 0.001 to 100 mg /kg/day, preferably from 0.01 mg/kg/day to 1 mg/kg/day. The compounds of the present invention may be administered in a single daily dose, or the total daily dose may be administered in divided doses, two, three, or more times per day. Where delivery is via transdermal forms, of course, administration is continuous.

### EXAMPLES

The present invention will be described as a form of examples, but they should by no means be construed as defining the metes and bounds of the present invention.

In the examples below, all quantitative data, if not stated otherwise, relate to percentages by weight.

Mass spectra were obtained using electrospray (ES) ionization techniques (micromass Platform LC). Melting points are uncorrected. Liquid Chromatography-Mass spectroscopy (LC-MS) data were recorded on a Micromass Platform LC with Shimadzu Phenomenex ODS column (4.6 mm×30 mm) flushing a mixture of acetonitrile-water (9:1 to 1:9) at 1 ml/min of the flow rate. TLC was performed on a precoated silica gel plate (Merck silica gel 60 F-254). Silica gel (WAKO-gel C-200 (75-150 μm)) was used for all column chromatography separations. All chemicals were reagent grade and were purchased from Sigma-Aldrich, Wako pure chemical industries, Ltd., Tokyo kasei kogyo co. Ltd., Arch cooperation.

All starting materials are commercially available or can be prepared using methods cited in the literature.

The effect of the present compounds were examined by the following assays and pharmacological tests.

[Measurement of Capsaicin-Induced Ca²⁺ Influx in the Human VR1-Transfected CHO Cell Line] (Assay 1)

(1) Establishment of the Human VR1-CHOluc9aeq Cell Line

Human vanilloid receptor (hVR1) cDNA was cloned from libraries of axotomized dorsal root ganglia (WO2000/29577). The cloned hVR1 cDNA was constructed with pcDNA3 vector and transfected into a CHOluc9aeq cell line. The cell line contains aequorin and CRE-luciferase reporter genes as read-out signals. The transfectants were cloned by limiting dilution in selection medium (DMEM/F12. medium (Gibco BRL) supplemented with 10% FCS, 1.4 mM Sodium pyruvate, 20 mM HEPES, 0.15% Sodium bicarbonate, 100 U/ml penicillin, 100 μg/ml strept mycin, 2 mM glutamine, non-essential amino acids and 2 mg/ml G418). Ca²⁺ influx was examined in the capsaicin-stimulated clones. A high responder clone was selected and used for further experiments in the project. The human VR1-CHOluc9aeq cells were maintained in the selection medium and passaged every 3-4 days at 1-2.5×10⁵ cells/flask (75 mm²).

(2) Measurement of Ca²⁺ Influx Using FDSS-3000

Human VR1-CHOluc9aeq cells were suspended in a culture medium which is the same as the selection medium except for G418 and seeded at a density of 1,000 cells per well into 384-well plates (black walled clear-base/Nalge Nunc International). Following the culture for 48 hrs the medium was changed to 2 μM Fluo-3 AM (Molecular Probes) and 0.02% Puronic F-127 in assay buffer (Hank's balanced salt solution (HBSS), 17 mM HEPES (pH7.4), 1 mM Probenecid, 0.1% BSA) and the cells were incubated for 60 min at 25° C. After washing twice with assay buffer the cells were incubated with a test compound or vehicle for 20 min at 25° C. Mobilization of cytoplasmic Ca²⁺ was measured by FDSS-3000 (λₑₓ=488 nm, λₑₘ=540 nm/Hamamatsu Photonics) for 60 sec after the stimulation with 10 nM capsaicin. Integral R was calculated and compared with controls.

[Measurement of the Capsaicin-Induced Ca²⁺ Influx in Primary Cultured Rat Dorsal Root Ganglia Neurons] (Assay 2)

(1) Preparation of Rat Dorsal Root Ganglia Neurons

New born Wister rats (5-11 days) were sacrificed and dorsal root ganglia (DRG) was removed. DRG was incubated with 0.1% trypsin (Gibco BRL) in PBS(−) (Gibco BRL) for 30 min at 37° C., then a half volume of fetal calf serum (FCS) was added and the cells were spun down. The DRG neuron cells were resuspended in Ham F12/5% FCS15% horse serum (Gibco BRL) and dispersed by repeated pipetting and passing through 70 μm mesh (Falcon). The culture plate was incubated for 3 hours at 37° C. to remove contaminating Schwann cells. Non-adherent cells were recovered and further cultured in laminin-coated 384 well plates. (Nunc) at 1×10⁴ cells/50 μl/well for 2 days in the presence of 50 ng/ml recombinant rat NGF (Sigma) and 50 μM 5-fluorodeoxyuridine (Sigma).

(2) Ca²⁺ Mobilization Assay

DRG neuron cells were washed twice with HBSS supplemented with 17 mM HEPES (pH 7.4) and 0.1% BSA. After incubating with 2 μM fluo-3AM (Molecular Probe), 0.02% PF127 (Gibco BRL) and 1 mM probenecid (Sigma) for 40 min at 37° C., cells were washed 3 times. The cells were incubated with VR1 antagonists or vehicle (dimethylsulphoxide) and then with 1 μM capsaicin in FDSS-6000 (λₑₓ=480 nm, λₑₘ=520 nm/Hamamatsu Photonics). The fluorescence changes at 480 nm were monitored for 2.5 min. Integral R was calculated and compared with controls.

[Organ Bath Assay to Measure the Capsaicin-Induced Bladder Contraction] (Assay 3)

Male Wistar rats (10 week old) were anesthetized with ether and sacrificed by dislocating the necks. The whole urinary bladder was excised and placed in oxygenated Modified Krebs-Henseleit solution (pH 7.4) of the following composition (112 mM NaCl, 5.9 mM KCl, 1.2 mM MgCl₂, 1.2 mM NaH₂PO₄, 2 mM CaCl₂, 2.5 mM NaHCO₃, 12 mM glucose). Contractile responses of the urinary bladder were studied as described previously [Maggi C A et al: Br. J. Pharmacol. 108: 801-805, 1993]. Isometric tension was recorded under a load of 1 g using longitudinal strips of rat detrusor muscle. Bladder strips were equilibrated for 60 min before each stimulation. Contractile response to 80 mM KCl was determined at 15 min intervals until reproducible responses were obtained. The response to KCl was used as an internal standard to evaluate the maximal response to capsaicin. The effects of the compounds were investigated by incubating the strips with compounds for 30 min prior to the stimulation with 1 μm capsaicin (vehicle: 80% saline, 10% EtOH, and 10% Tween 80). One of the preparations made from the same animal was served as a control while the others were used for evaluating compounds. Ratio of each capsaicin-induced contraction to the internal standard (i.e. KCl-induced contraction) was calculated and the effects of the test compounds on the capsaicin-induced contraction were evaluated.

[Measurement of Ca²⁺ Influx in the Human P2X1-Transfected CHO Cell Line]

(1) Preparation of the Human P2X1-Transfected CHOluc9aeq Cell Line

Human P2X1-transfected CHOluc9aeq cell line was established and maintained in Dulbecco's modified Eagle s medium (DMEM/F12) supplemented with 7.5% FCS, 20 mM HEPES-KOH (pH 7.4), 1.4 mM sodium pyruvate, 100 U/ml penicillin, 100 μg/ml streptomycin, 2 mM glutamine (Gibco BRL) and 0.5 Units/ml apyrase (grade I, Sigma). The suspended cells were seeded in each well of 384-well optical bottom black plates (Nalge Nunc International) at 3×10³/50 μl well. The cells were cultured for following 48 hrs to adhere to the plates.

(2) Measurement of the Intracellular Ca²⁺ Levels

P2X1 receptor agonist-mediated increases in cytosolic Ca²⁺ levels were measured using a fluorescent Ca²⁺ chelating dye, Fluo-3 AM (Molecular Probes). The plate-attached cells were washed twice with washing buffer (HBSS, 17 mM HEPES-KOH. (pH 7.4), 0.1% BSA and 0.5 units/ml apyrase), and incubated in 40 μl of loading buffer (1 μM Fluo-3 AM, 1 mM probenecid, 1 μM cyclosporin A, 0.01% pluronic (Molecular Probes) in washing buffer) for 1 hour in a dark place. The plates were washed twice with 40 μl washing buffer and 35 μl of washing buffer were added in each well with 5 μl of test compounds or 2′,3′-o-(2,4,6-trinitrophenyl) adenosine 5′-tiphosphate (Molecular Probes) as a reference. After further incubation for 10 minutes in dark 200 nM α,β-methylene ATP agonist was added to initiate the Ca²⁺ mobilization. Fluorescence intensity was measured by FDSS-6000 (λₑₓ=410 nm, λₑₘ=510 nm/Hamamatsu Photonics) at 250 msec intervals. Integral ratios were calculated from the data and compared with that of a control.

[Measurement of Capsaicin-Induced Bladder Contraction in Anesthetized Rats] (Assay 4)

(1) Animals

Female Sprague-Dawley rats (200˜250 g/Charles River Japan) were used.

(2) Catheter Implantation

Rats were anesthetized by intraperitoneal administration of urethane (Sigma) at 1.2 g/kg. The abdomen was opened through a midline incision, and a polyethylene catheter (BECTON DICKINSON, PE50) was implanted into the bladder through the dome. In parallel, the inguinal region was incised, and a polyethylene catheter (Hibiki, size 5) filled with 2 IU/ml of heparin (Novo Heparin, Aventis Pharma) in saline (Otsuka) was inserted into a common iliac, artery.

(3) Cystometric Investigation

The bladder catheter was connected via T-tube to a pressure transducer (Viggo-Spectramed Pte Ltd, DT-XXAD) and a microinjection pump (TERUMO). Saline was infused at room temperature into the bladder at a rate of 2.4 ml/hr. Intravesical pressure was recorded continuously on a chart pen recorder (Yokogawa). At least three reproducible micturition cycles, corresponding to a 20-minute period, were recorded before a test compound administration and used as baseline values.

(4) Administration of Test Compounds and Stimulation of Bladder With Capsaicin

The saline infusion was stopped before administrating compounds. A testing compound dissolved in the mixture of ethanol, Tween 80 (ICN Biomedicals Inc.) and saline (1:1:8, v/v/v) was administered intraarterially at 10 mg/kg. 2 min after the administration of the compound 10 μg of capsaicin (Nacalai Tesque) dissolved in ethanol was administered intraarterially.

(5) Analysis of Cystometry Parameters

Relative increases in the capsaicin-induced intravesical pressure were analyzed from the cystometry data. The capsaicin-induced bladder pressures were compared with the maximum bladder pressure during micturition without the capsaicin stimulation. The testing compounds-mediated inhibition of the increased bladder pressures was evaluated using Student's t-test. A probability level less than 5% was accepted as significant difference.

Results of IC₅₀ of capsaicin-induced Ca²⁺ influx in the human VR1-transfected CHO cell line are shown in Examples and tables of the Examples below. The data corresponds to the compounds as yielded by solid phase synthesis and thus to levels of purity of about 40 to 90%. For practical reasons, the compounds are grouped in four classes of activity as follows:
IC₅₀=A 0.1 μM<B 0.5 μM<C 1 μM<D

The compounds of the present invention also show excellent selectivity, and strong activity in other assays (2)-(4) described above.

### Example 1

### N-(1,3-benzothiazol-6-yl)-N′-[4-chloro-3-(trifluoromethyl)phenyl]urea

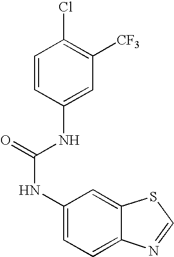

This example was performed according to the general method A.

To a stirred solution of 1,3-benzothiazol-6-amine (50.0 mg, 0.33 mmol) in 1,4-dioxane (5.0 ml) was added a solution of 1-chloro-4-isocyanato-2-(trifluoromethyl)-benzene (88.5 mg, 0.40 mmol) in 1,4-dioxane (1.0 ml) at room temperature. A catalytic amount (2 drops) of pyridine was added and the reaction mixture was warmed to 50° C., and stirred for 20 hrs at the same temperature. The solvent was removed under reduced pressure, and the residue was washed with ⁱPr₂O/MeOH to give N-(1,3-benzothiazol-6-yl)-N′-[4-chloro-3-(trifluoromethyl)phenyl]urea as a grayish powder:

mp225-228° C.; Molecular weight 371.77 MS (M+H): 372 Activity grade: A

### Example 2

### N-(1,1′-biphenyl-3-yl)-N′-(1H-indol-4-yl)urea

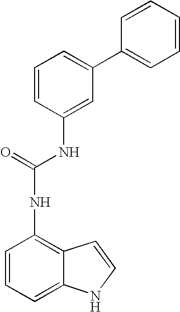

This example was performed according to the general method B.

To a suspension of 1,1′-carbonyldi(1,2,4-triazole) (62.1 mg, 0.38 mmol) in THF (5.0 ml), was added dropwise a solution of 1H-indol-4-amine (50.0 mg, 0.38 mmol) in THF (1.0 ml) at room temperature. The resulting suspension was stirred for 1 hour. 1,1′-biphenyl-3-amine (64.0 mg, 0.4mmol) was added to the suspension at room temperature. The reaction mixture was stirred at 50° C. for 15 hrs. After cooling to room temperature, the solvent was removed under reduced pressure. The residue was dissolved in a mixture of ethyl acetate and ethanol (1:1), and was passed through a silicagel short cartridge (1 g Si/6 ml). The cartridge was washed with a mixture of ethyl acetate and ethanol (1:1). The combined filtrates were concentrated to give a solid.

The crude product was washed with a mixture of isopropanol and isopropyl ether to give N-(1,1′-biphenyl-3-yl)-N′-(1H-indol-4-yl)urea as a powder (59.0 mg, 48%).

m.p. 213-215° C.; Molecular weight 327.39 MS (M+H): 328 Activity grade: A

According to procedures similar to the examples above, the following compounds were synthesized and tested. The compounds listed below can be prepared by either of the methods A, B or C.

TABLE 1

| | | | | melting | hVR1 |
|---|---|---|---|---|---|
| Ex. NO. | MOLSTRUCTURE | MW | MS | point | class |
| 3 | 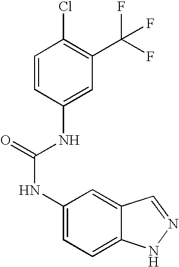 | 354.7214 | 355 | >250 | A |
| 4 | 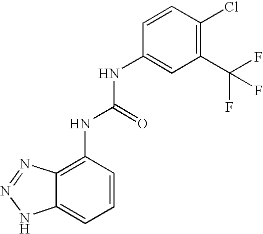 | 355.7089 | 356 | 232-235 | B |
| 5 | 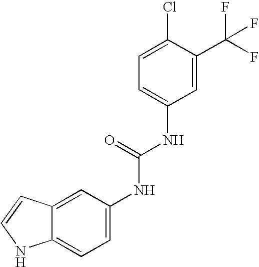 | 353.7338 | 354 | 234-235 | B |
| 3 | 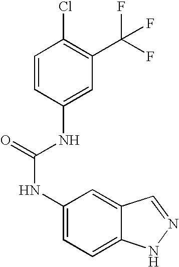 | 354,72135 | 355 | >250 | A |
| 4 | 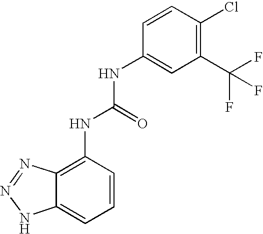 | 355,70893 | 356 | 232-235 | B |
| 5 | 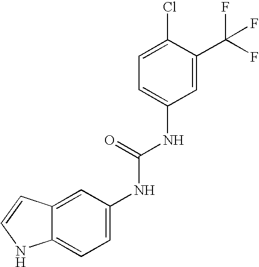 | 353,73377 | 354 | 234-235 | B |
| 6 | 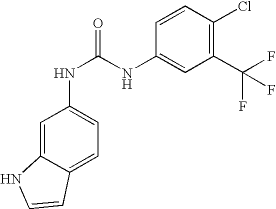 | 353,73377 | 354 | 245-248 | B |
| 7 | 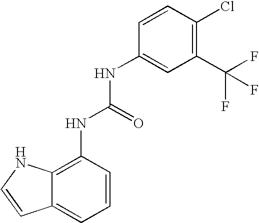 | 353,73377 | 354 | 229-233 | A |
| 8 | 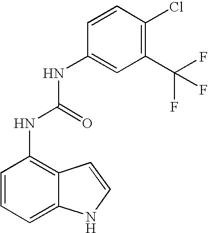 | 353,73377 | 354 | 230-233 | A |
| 9 | 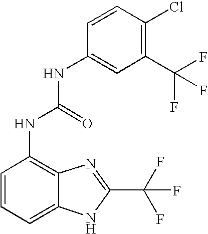 | 422,71973 | 423 | 165-169 | A |
| 10 | 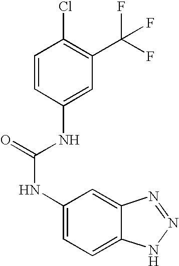 | 355,70893 | 356 | >250 | B |
| 11 | 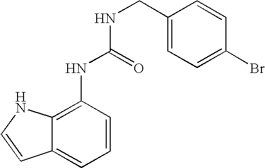 | 344,21348 | 344 | 205-208 | A |
| 12 | 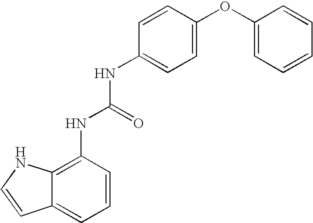 | 343,38854 | 344 | 216-218 | B |
| 13 | 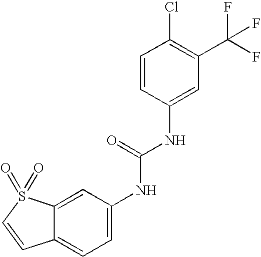 | 402,7819 | 403 | ND | A |
| 14 | 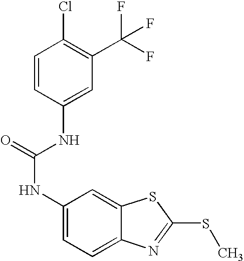 | 417,86177 | 418 | 236-238 | B |
| 15 | 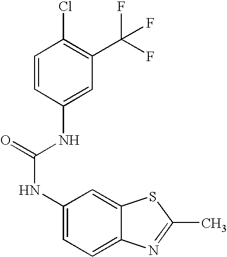 | 385,79777 | 386 | 234-235 | B |
| 16 | 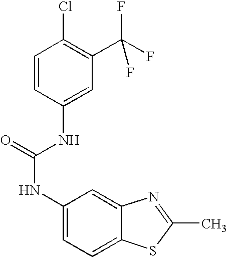 | 385,79777 | 386 | 152-155 | A |
| 17 | 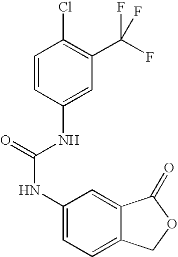 | 370,7179 | 371 | >250 | B |
| 18 | 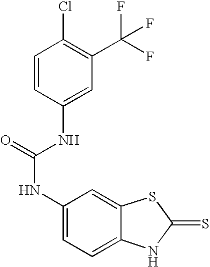 | 403,83468 | 404 | >250 | C |
| 19 | 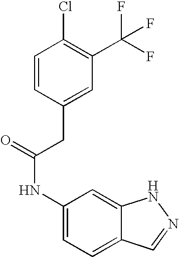 | 354,72135 | 355 | >250 | C |
| 20 | 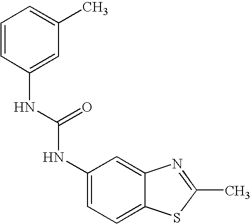 | 297,38145 | 298 | 200-202 | A |
| 21 | 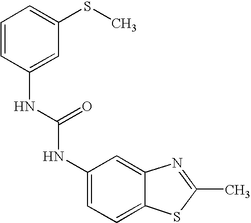 | 329,44545 | 330 | 225-227 | B |
| 22 | 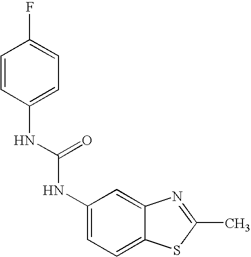 | 301,34479 | 302 | 241-242 | A |
| 23 | 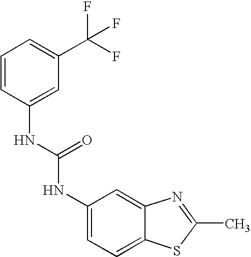 | 351,35274 | 352 | 229-231 | A |
| 24 | 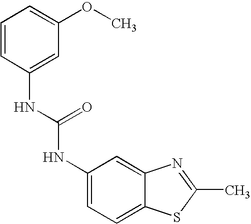 | 313,38085 | 314 | 199-201 | B |
| 25 | 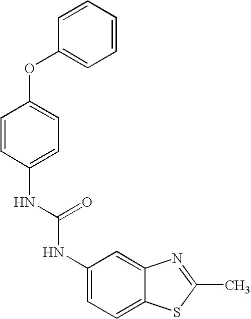 | 375,45254 | 376 | 228-229 | A |
| 26 | 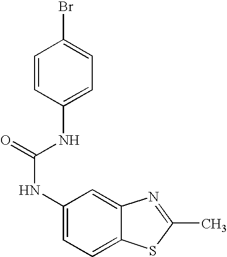 | 362,25039 | 364 | >250 | A |
| 27 | 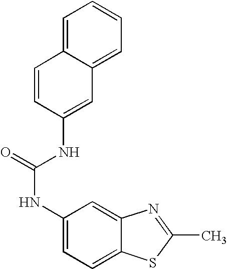 | 333,4149 | 334 | >250 | A |
| 28 | 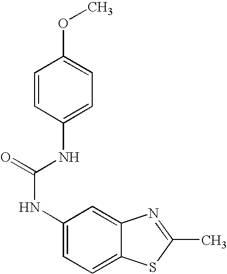 | 313,38085 | 314 | 215-217 | B |
| 29 | 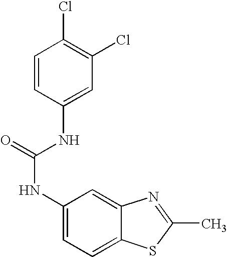 | 352,24442 | 352 | 231 233 | A |
| 30 | 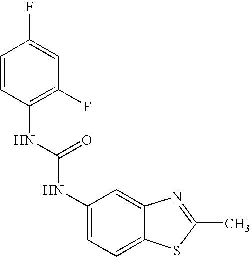 | 319,33522 | 320 | 243-244 | A |
| 31 | 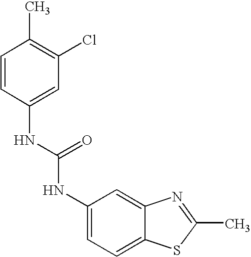 | 331,82648 | 332 | 230-232 | A |
| 32 | 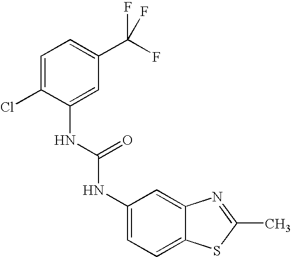 | 385,79777 | 386 | 240-241 | A |
| 33 | 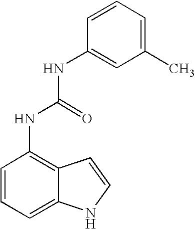 | 265,31745 | 266 | 237-239 | B |
| 34 | 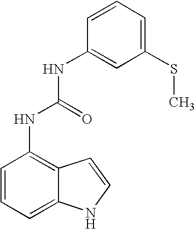 | 297,38145 | 298 | 198-201 | B |
| 35 | 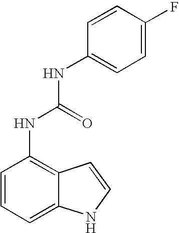 | 269,28079 | 270 | 239-241 | B |
| 36 | 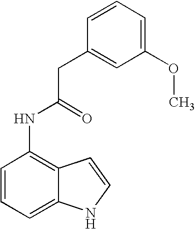 | 281,31685 | 282 | 219-221 | B |
| 37 | 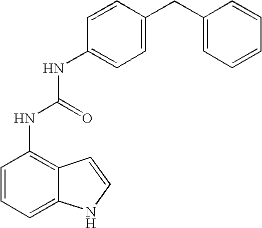 | 343,38854 | 344 | 212-214 | B |
| 38 | 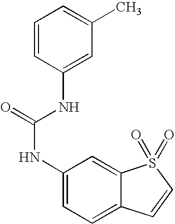 | 314,36558 | 315 | 219-222 | C |
| 39 | 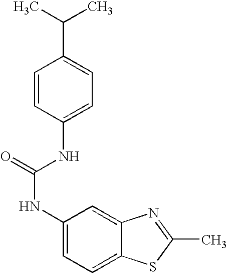 | 325,43563 | 326 | 208-210 | A |
| 40 | 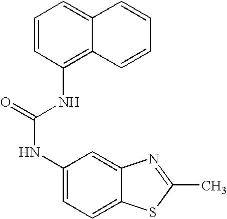 | 333.4149 | 334 | >250 | A |
| 41 | 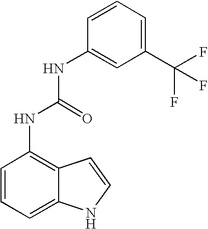 | 319,28874 | 320 | 211-213 | A |
| 42 | 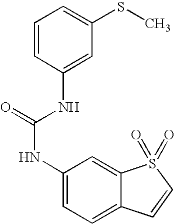 | 346,42958 | 347 | 212-213 | B |
| 43 | 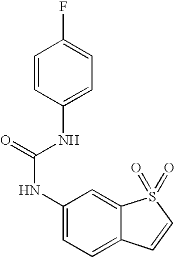 | 318,32892 | 319 | 242-243 | C |
| 44 | 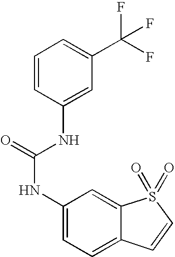 | 368,33687 | 369 | >250 | 8 |
| 45 | 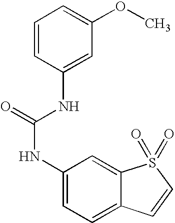 | 330,36498 | 331 | 206-208 | C |
| 46 | 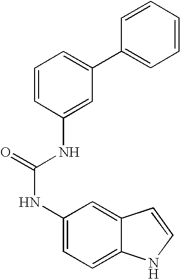 | 327,38914 | 328 | 204-206 | B |
| 47 | 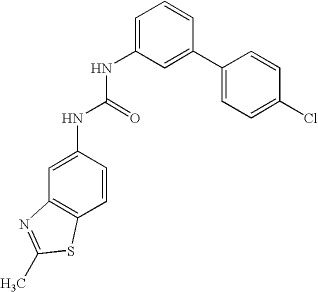 | 393,89817 | | | B |
| 48 | 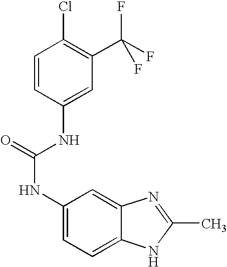 | 368,74844 | 369 | 162-166 | C |
| 49 | 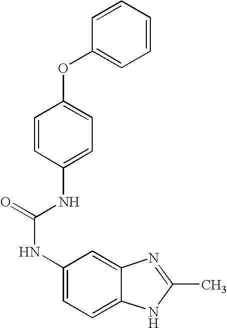 | 358,40321 | 359 | 243-245 | C |
| 50 | 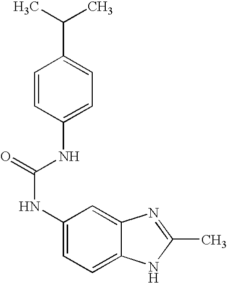 | 308,3863 | 309 | >250 | C |
| 51 | 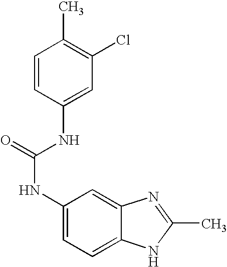 | 314,77715 | 315 | 200-204 | C |
| 52 | 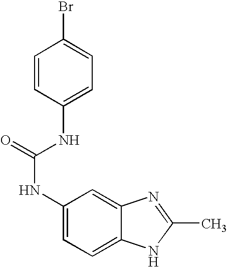 | 345,20106 | 347 | >250 | C |
| 53 | 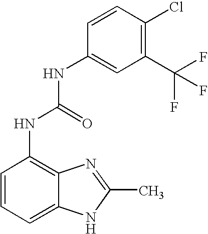 | 368,74844 | 369 | 189-191 | A |
| 54 | 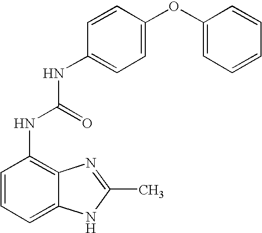 | 358,40321 | 359 | 223-225 | A |
| 55 | 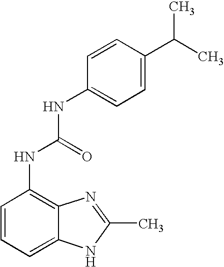 | 308,3863 | 309 | 216-218 | B |
| 56 | 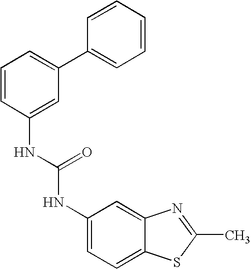 | 359,45314 | 360 | 216-219 | B |
| 57 | 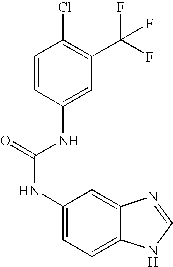 | 354,72135 | 355 | 218-220 | B |
| 58 | 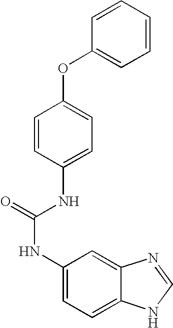 | 344,37612 | 345 | 235-237 | B |
| 59 | 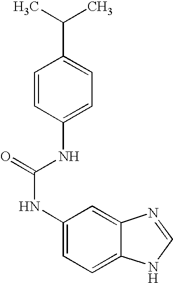 | 294,35921 | 295 | 226-229 | C |
| 60 | 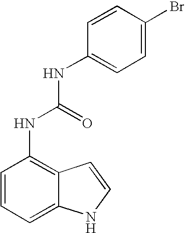 | 330,18639 | 332 | 238-240 | B |
| 61 | 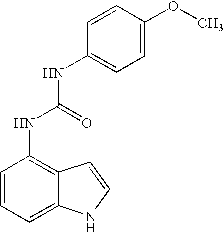 | 281,31685 | 282 | 230-232 | C |
| 62 | 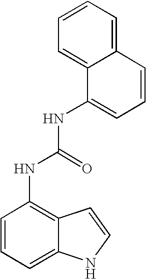 | 301,3509 | 302 | >250 | A |
| 63 | 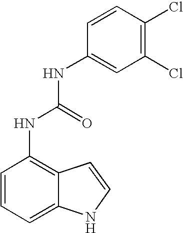 | 320,18042 | 320 | 244-245 | A |
| 64 | 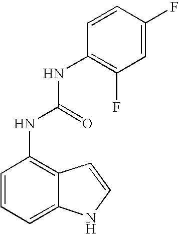 | 287,27122 | 288 | 247-248 | C |
| 65 | 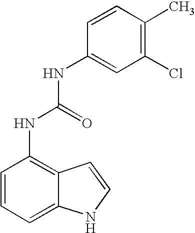 | 299,76248 | 300 | 246-247 | A |
| 66 | 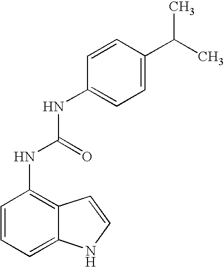 | 293,37163 | 294 | 222-224 | A |
| 67 | 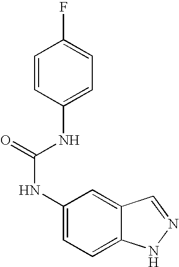 | 270,26837 | 271 | >250 | C |
| 68 | 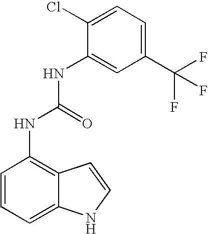 | 353,73377 | | 211-213 | A |
| 69 | 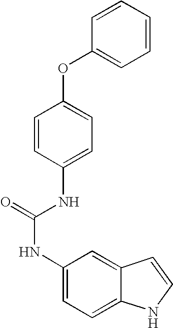 | 343,38854 | 344 | 231-233 | B |
| 70 | 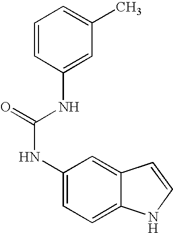 | 265,31745 | 266 | 250 | C |
| 71 | 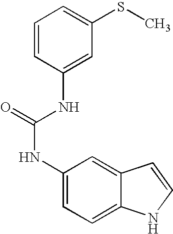 | 297,38145 | 298 | 236-239 | B |
| 72 | 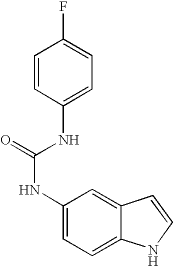 | 269,28079 | 270 | 243-245 | C |
| 73 | 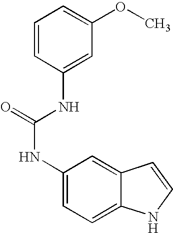 | 281,31685 | 282 | 227-229 | C |
| 74 | 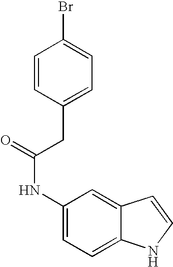 | 330,18639 | 332 | >250 | C |
| 75 | 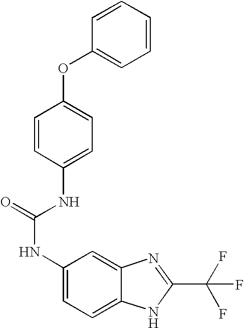 | 412,3745 | 413 | 239-241 | C |
| 76 | 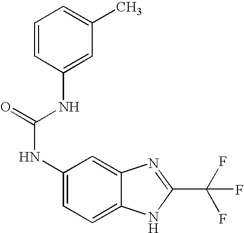 | 334,30341 | 335 | 245-247 | C |
| 77 | 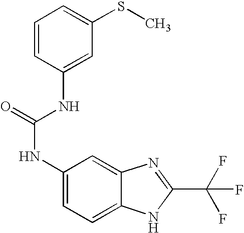 | 366,36741 | 367 | 226-228 | C |
| 78 | 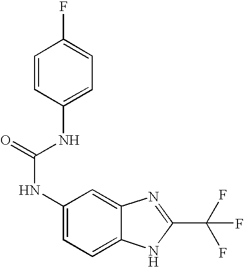 | 338,26675 | 339 | 242-243 | C |
| 79 | 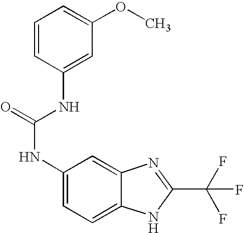 | 350,30281 | 351 | 233-237 | C |
| 80 | 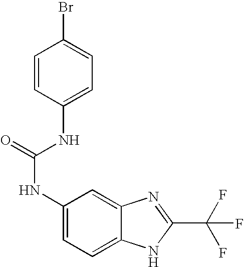 | 399,17235 | 401 | >250 | C |
| 81 | 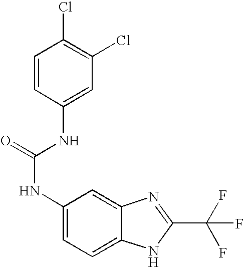 | 389,16638 | 389 | 240-242 | C |
| 82 | 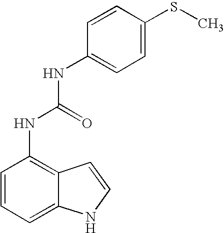 | 297,38145 | 298 | 228-231 | C |
| 83 | 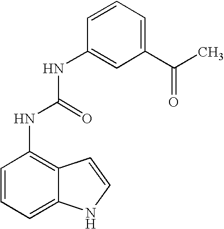 | 293,328 | 294 | 205-207 | C |
| 84 | 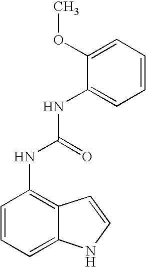 | 281,31685 | 282 | 208-209 | C |
| 85 | 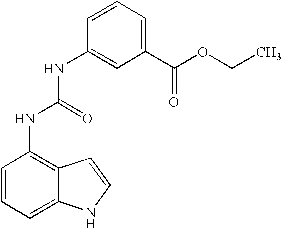 | 323,35449 | 324 | 194-196 | A |
| 86 | 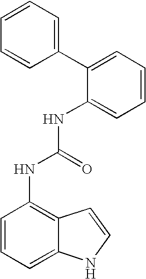 | 327,38914 | 328 | 104-106 | C |
| 87 | 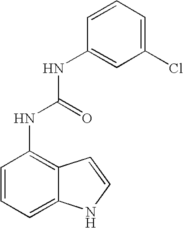 | 285,73539 | 286 | 238-239 | B |
| 88 | 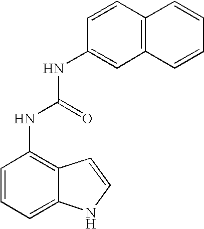 | 301,3509 | 302 | 242-243 | B |
| 89 | 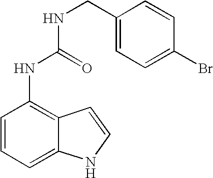 | 344,21348 | 346 | 199-202 | A |
| 90 | 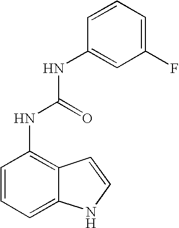 | 269,28079 | 270 | 225-226 | C |
| 91 | 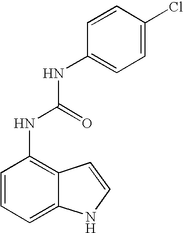 | 285,73539 | 286 | 247-248 | B |
| 92 | 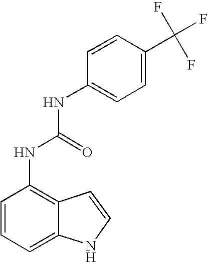 | 319,28874 | 320 | 242-243 | B |
| 93 | 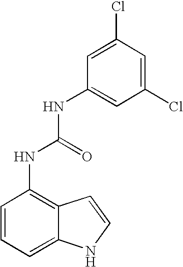 | 320,18042 | 320 | 262-263 | B |
| 94 | 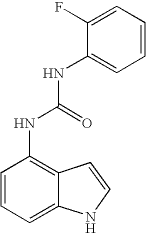 | 269,28079 | 270 | 244-246 | C |
| 95 | 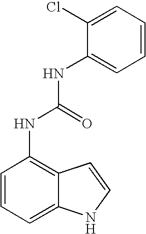 | 285,73539 | 286 | 244-246 | B |
| 96 | 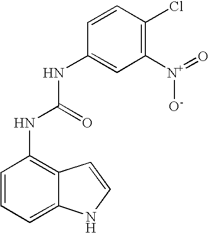 | 330,73292 | 331 | 233-235 | B |
| 97 | 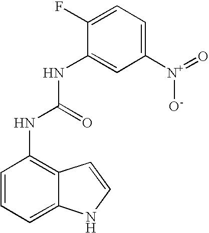 | 314,27832 | 315 | 261-263 | C |
| 98 | 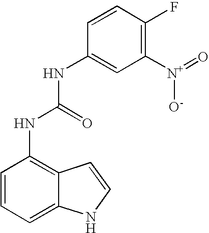 | 314,27832 | 315 | 248-251 | B |
| 99 | 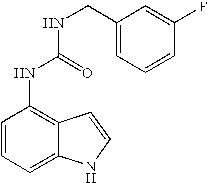 | 283,30788 | 284 | 190-192 | C |
| 100 | 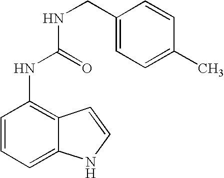 | 279,34454 | 280 | 223 | B |
| 101 | 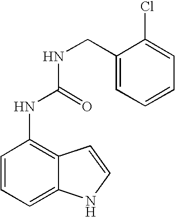 | 299,76248 | 300 | 237-238 | B |
| 102 | 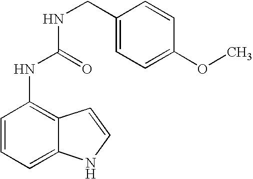 | 295,34394 | 298 | 201-202 | C |
| 103 | 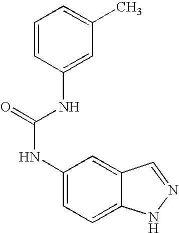 | 266,30503 | 267 | ND | C |
| 104 | 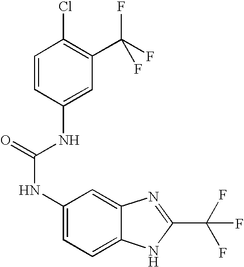 | 422,71973 | 423 | ND | C |
| 105 | 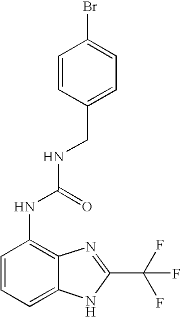 | 413,19944 | 415 | ND | C |
| 106 | 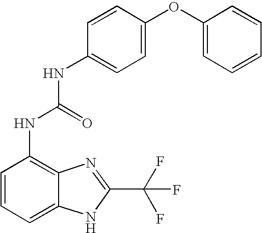 | 412,3745 | 413 | ND | B |

1) a medicament comprising a urea derivative of the formula (I), its tautomeric or stereoisomeric form, or a salt thereof as an active ingredient: 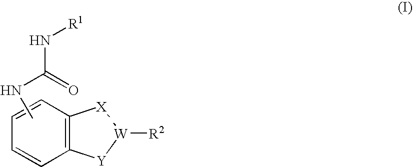
wherein
R¹ is C₁₋₆ alkyl substituted by phenyl or thienyl (in which said phenyl and thienyl are substituted by R¹¹, R¹², and R¹³), C₃₋₈ cycloalkyl optionally fused by benzene, thienyl, quinolyl, carbazolyl of which N—H is substituted by N—R¹¹, 1,2-oxazolyl substituted by R¹¹, naphthyl substituted by R¹⁴ and R¹⁵ phenyl substituted by R¹¹, R¹², and R¹³, phenyl fused by C₄₋₈ cycloalkyl or saturated or unsaturated C₄₋₈ heterocyclic ring having one or two hetero atoms selected from the group consisting of N, O, S, and SO₂,
wherein said cycloalkyl and heterocyclic ring are optionally substituted by R¹¹,
in which
R¹¹, R¹² and R¹³ are different or identical and represent hydrogen, halogen, oxo, nitro, carboxyl, C₁₋₆ alkyl optionally substituted by hydroxy or mono-, di-, or tri-halogen, carbamoyl, C₁₋₆ alkyl-carbamoyl, C₁₋₆ alkoxy optionally substituted by mono-, di-, or tri-halogen, C₁₋₆ alkoxycarbonyl, amino, C₁₋₆ alkylamino, di(C₁₋₆ alkyl)amino, morpholino, benzyl, phenoxy, mono-, di-, or tri-halogen substituted phenoxy, C₁₋₆ alkylthio, C₁₋₆ alkanoyl, C₁₋₆ alkanoylamino, C₁₋₆ alkyl substituted 4,5-dihydro-1,3-oxazolyl, 1,2,3-thiadiazolyl, phenyl optionally substituted by one to three substituents,
in which the substituents are each different or identical and selected from the group consisting of hydrogen, halogen, C₁₋₆ alkoxy, C₁₋₆ alkyl C₁₋₆ alkanoyl, and carboxy, or
the substituent represented by the formula —SO₂—N—R¹¹¹
wherein
R¹¹¹ represents hydrogen, 5-methyl-isoxazole, or 2,4 dimethylpyrimidine;
R¹⁴ is hydrogen, hydroxy, or C₁₋₆ alkoxy;
R¹⁵ is hydrogen, hydroxy, or C₁₋₆ alkoxy;
X, Y, and W are different or identical represent C, CH, CH₂, C(O), N, NH, S, O, SO or SO₂;
the dashed line between X and W represents a single bond or a double bond;
R² is selected from the group consisting of hydrogen, methyl, hydroxy, mercapto, trifluoromethyl, and methylthio, or
is absent;
with the proviso that
if the bond between X—W is a double,
X is N or CH;
W is N or C; and
Y is selected from the group consisting of NH, S, O, CH₂, SO, and SO₂;
with the proviso that when W is N,R² is absent;
if the bond between X—W is a single,
X and Y independently represent CH₂, CO, NH, S, O, SO, or SO₂;
W is N, CH, S, O, SO or SO₂;
with the proviso that when W is S, O, SO or SO₂, R² is absent.

2) The medicament comprising a urea derivative of the formula (I), as claimed in claim 1, wherein
R¹ is 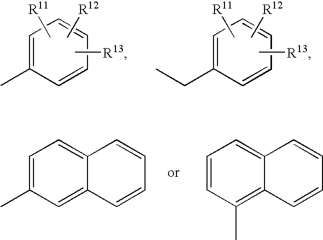
wherein
R¹¹, R¹², and R¹³ are different or identical and represent hydrogen, halogen, nitro, carboxyl, C₁₋₆ alkyl optionally substituted by hydroxy or mono-, di-, or tri-halogen, C₁₋₆ alkoxy optionally substituted by mono-, di-, or tri-halogen, C₁₋₆ alkoxycarbonyl, carbamoyl, C₁₋₆ alkylcarbamoyl, amino, C₁₋₆ alkylamino, di(C₁₋₆ alkyl)amino, morpholino, phenyl, benzyl, phenoxy, mono-, di-, or tri-halogen substituted phenoxy, mono-, di-, or tri-halogen substituted phenyl, C₁₋₆ alkylthio, C₁₋₆ alkanoyl, C₁₋₆ alkanoylamino, or the substituent represented by the formula —SO₂—N—R¹¹¹
wherein
R¹¹¹ is hydrogen, 5-methyl-isoxazole, or 2,4-dimethyl-pyrimidine.

3) A medicament comprising a urea derivative of the formula (1), as claimed in claim 1,
wherein
R¹ is 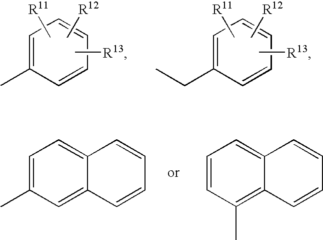
wherein
R¹¹, R¹², and R¹³ are different or identical and represent hydrogen, fluoro, chloro, bromo, methyl, isopropyl, methoxy, nitro, ethoxycarbonyl, phenyl, phenoxy, 4-chlorophenyl, methylthio, acetyl, or trifluoromethyl.

4) A medicament comprising a urea derivative of the formula (I), as claimed in claim 1,
wherein 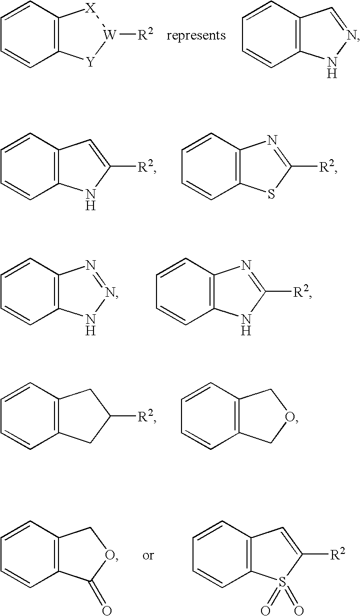
wherein
R² is hydrogen, methyl, hydroxy, mercapto, trifluoromethyl, or methylthio.

5) A medicament comprising a urea derivative of the formula (I), as claimed in claim 1,
wherein
R² is hydrogen, methyl, trifluoromethyl, or methylthio.

6) The medicament as claimed in claim 1, wherein said urea derivative of the formula (I) its tautomeric or stereoisomeric form, or a salt thereof is selected from the group consisting of
N-[4-chloro-3-(trifluoromethyl)phenyl]-N′-(1H-indazol-5-yl)urea;
N-[4-chloro-3-(trifluoromethyl)phenyl]-N′-(1H-indol-7-yl)urea;
N-[4-chloro-3-(trifluoromethyl)phenyl]-N′-(1H-indol-4-yl)urea;
N-[4-chloro-3-(trifluoromethyl)phenyl]-N′-[2-(trifluoromethyl)-1H-benzimidazol-4-yl]urea;
N-(4-bromobenzyl)-N′-(1H-indol-7-yl)urea;
N-[4-chloro-3-(trifluoromethyl)phenyl]-N′-(1,1-dioxido-1-benzothien-6-yl)urea;
N-(1,3-benzothiazol-6-yl)-N′-[4chloro-3-(trifluoromethyl)phenyl]urea;
N-[4-chloro-3-(trifluoromethyl)phenyl]-N′-(2-methyl-1,3-benzothiazol-5-yl)urea;
N-(2-methyl-1,3-benzothiazol-5-yl)-N′-(3-methylphenyl)urea;
N-(4-fluorophenyl)-N′-(2-methyl-1,3-benzothiazol-5-yl)urea;
N-(2-methyl-1,3-benzothiazol-5-yl)-N′-[3-(trifluoromethyl)phenyl]urea;
N-(2-methyl-1,3-benzothiazol-5-yl)-N′-(4-phenoxyphenyl)urea;
N-(4-bromophenyl)-N′-(2-methyl-1,3-benzothiazol-5-yl)urea;
N-(2-methyl-1,3-benzothiazol-5-yl)-N′-(2-naphthyl)urea;
N-(3,4-dichlorophenyl)-N′-(2-methyl-1,3-benzothiazol-5-yl)urea;
N-(2,4-difluorophenyl)-N′-(2-methyl-1,3-benzothiazol-5-yl)urea;
N-(3-chloro-4-methylphenyl)-N′-(2-methyl-1,3-benzothiazol-5-yl)urea;
N-[2-chloro-5-(trifluoromethyl)phenyl]-N-(2-methyl-1,3-benzothiazol-5-yl)urea;
N-(4-isopropylphenyl)-N′-(2-methyl-1,3-benzothiazol-5-yl)urea;
N-(2-methyl-1,3-benzothiazol-5-yl)-N′-(1-naphthyl)urea;
N-(1H-indol4-yl)-N′-[3-(trifluoromethyl)phenyl]urea;
N-(1,1′-biphenyl-3-yl)-N′-(1H-indol-4-yl)urea;.
N-[4-chloro-3-(trifluoromethyl)phenyl]-N′-(2-methyl-1H-benzimidazol-4-yl)urea;
N-(2-methyl-1H-benzimidazol-4-yl)-N′-(4-phenoxyphenyl)urea;
N-(1H-indol-4-yl)-N′-(1-naphthyl)urea;
N-(3,4-dichlorophenyl)-N′-(1H-indol-4-yl)urea;
N-(3-chloro-4-methylphenyl)N′-(1H-indol-4-yl)urea;
N-(1H-indol-4-yl)-N′-(4-isopropylphenyl)urea;
N-(4-fluorophenyl)-N′-(1H-indazol-5-yl)urea;
N-[2-chloro-5-(trifluoromethyl)phenyl]-N′-(1H-indol-4-yl)urea;
ethyl 3-{[(1H-indol-4-ylamino)carbonyl]amino}benzoate; and
N-(4-bromobenzyl)-N-(1H-indol-4-yl)urea.

7) The medicament as claimed in claim 1 further comprising one or more pharmaceutically acceptable excipients.

8) The medicament as claimed in claim 1, wherein said urea derivative of the formula (I), its tautomeric or stereoisomeric form, or a salt thereof is a VR1 antagonist.

9) The medicament as claimed in claim 1, wherein said urea derivative of the formula (I), its tautomeric or stereoisomeric form, or a salt thereof is effective for treating or preventing a disease selected from the group consisting of urge urinary incontinence, overactive bladder, chronic pain, neuropathic pain, postoperative pain, rheumatoid arthritic pain, neuralgia, neuropathies, algesia, nerve injury, ischaemia, neurodegeneration, stroke, incontinence and inflammatory disorders.

10) A method for treating or preventing disorder or disease associated with VR1 activity in a human or animal subject, comprising administering to said subject a therapeutically effective amount of the medicament as claimed in claim 1.

11) The method of claim 10, wherein said disorder or disease is a urological disorder or disease.

12) The method of claim 10, wherein said disorder or disease is selected from the group consisting of urinary incontinence, overactive bladder, chronic pain, neuropathic pain, postoperative pain, rheumatoid arthritic pain, neuralgia, neuropathies, algesia, nerve injury, ischaemia, neurodegeneration, stroke, incontinence and inflammatory disorders.

13) The method of claim 10, wherein said urea derivative, its tautomeric or stereoisomeric form, or a physiologically acceptable salt thereof is administered with one or more pharmaceutically acceptable excipients.

14) Process for controlling urological disorders in humans and animals by administration of a VR1-antagonisticly effective amount of at least one compound according to any of claims 1.
